(19) United States
(12) Patent Application Publication
(10) Pub. No.: US 2024/0268836 A1
(43) Pub. Date: Aug. 15, 2024

(54) BONE CUTTING GUIDE SYSTEMS AND METHODS

(71) Applicant: Treace Medical Concepts, Inc., Ponte Vedra Beach, FL (US)

(72) Inventors: Paul Dayton, Ankeny, IA (US); Robert D. Santrock, Morgantown, WV (US); Daniel J. Hatch, Greeley, CO (US); W. Bret Smith, Lexington, SC (US); Carlos Eduardo Gil, Memphis, TN (US); Sean F. Scanlan, Jacksonville, FL (US); Joe William Ferguson, Ponte Vedra Beach, FL (US); F. Barry Bays, Collierville, TN (US); John T. Treace, Ponte Vedra Beach, FL (US)

(21) Appl. No.: 18/645,205

(22) Filed: Apr. 24, 2024

Related U.S. Application Data
(63) Continuation of application No. 18/487,548, filed on Oct. 16, 2023.
Application No. 18/487,548 is a continuation of application No. 17/118,597, filed on Dec. 10, 2020, now Pat. No. 11,786,257.
Application No. 17/118,597 is a continuation of application No. 16/792,880, filed on Feb. 17, 2020, now Pat. No. 10,888,335.
Application No. 16/792,880 is a continuation of application No. 16/593,153, filed on Oct. 4, 2019, now Pat. No. 10,561,426.
Application No. 16/593,153 is a continuation of application No. 15/603,056, filed on May 23, 2017, now Pat. No. 10,603,046.
Application No. 15/603,056 is a continuation of application No. 14/990,574, filed on Jan. 7, 2016, now Pat. No. 9,687,250.
(60) Provisional application No. 62/100,641, filed on Jan. 7, 2015.

(51) Int. Cl. A61B 17/15; A61B 17/16

(52) U.S. Cl. CPC A61B 17/151; A61B 17/15; A61B 17/1682

(57) ABSTRACT

A bone cutting guide may include a support that contains a shaft movable relative to the support. The shaft may carry a guide member having one or more cut guides through which a clinician inserts a cutting member to cut bone positioned under the guide cut guides. In operation, a clinician may fixate the support of the bone cutting guide to a bone and translate the guide member until the one or more cut guides are positioned at a desired cut location. The clinician may then perform a cut through the cut guide. In some examples, the bone cutting guide includes additional components, such as bridging member or secondary cut guide, to provide additional functionality.

### RELATED APPLICATION

This application is a continuation of U.S. patent application Ser. No. 18/487,548, filed Oct. 16, 2023, which is continuation of U.S. patent application Ser. No. 17/118,597, filed Dec. 10, 2020 and issued as U.S. Pat. No. 11,786,257 on Oct. 17, 2023, which is a continuation of U.S. patent application Ser. No. 16/792,880, filed Feb. 17, 2020 and issued as U.S. Pat. No. 10,888,335 on Jan. 12, 2021, which is a continuation of U.S. patent application Ser. No. 16/593,153, filed Oct. 4, 2019 and issued as U.S. Pat. No. 10,561,426 on Feb. 18, 2020, which is a continuation of U.S. patent application Ser. No. 15/603,056, filed May 23, 2017 and issued as U.S. Pat. No. 10,603,046 on Mar. 31, 2020, which is a continuation of U.S. patent application Ser. No. 14/990,574, filed Jan. 7, 2016 and issued as U.S. Pat. No. 9,687,250 on Jun. 27, 2017, which in turn claims the benefit of U.S. Provisional Application Ser. No. 62/100,641, filed Jan. 7, 2015. The entire contents of each of these applications are hereby incorporated by reference.

### TECHNICAL FIELD

This disclosure relates generally to devices and methods for positioning and cutting bones.

### BACKGROUND

Bones, such as the bones of a foot, may be anatomically misaligned. In certain circumstances, surgical intervention is required to correctly align the bones to reduce patient discomfort and improve patient quality of life.

### SUMMARY

In general, this disclosure is directed to bone cutting guide systems and techniques for cutting bones. In some examples, a bone cutting guide includes a main body, or support, that houses a shaft that can translate relative to the main body. The shaft may have a main guide member positioned on the end of the shaft. The main guide member may define opposed guide surfaces configured to receive a cutting member. For example, the cutting member may be inserted between the opposed guide surfaces and bounded within a range of movement by the guide surfaces, causing the cutting member to be directed at a cutting location under the guide surfaces. Additionally or alternatively, the main guide member may define a single cutting surface/plane. The cutting surface/plane may be a surface against which a clinician can position a cutting member and then guide the cutting member along the cutting surface/plane to perform a cutting operation.

The main body of the bone cutting guide can include fixation members, such as fixation pins or apertures, that allow the main body to be fixated on or adjacent a bone to be cut. For example, in use, a clinician may fixate the main body to a bone (e.g., a first metatarsal). Thereafter, the clinician may translate the main guide member having at least one cutting guide surface (e.g., opposed cutting guide surfaces) relative to the fixed main body. The clinician can translate the main guide member by sliding or rotating the shaft housed within the main body, e.g., causing the distal end of the shaft and main guide member carried thereon away from or towards the main body. Once suitably positioned, the clinician may or may not lock the location of the shaft and perform one or more cuts through the guide surfaces of the main guide member.

In some configurations, the bone cutting guide also includes a bridge component that can form a bridge over a section of bone, such as a joint between adjacent bones (e.g., first metatarsal-medial cuneiform joint). For example, the bridge component may have a proximal end that is attachable to the main guide member carried on the shaft attached to the main body and a distal end separated by one or more rails. The proximal end may be insertable between the opposed cutting guide surfaces of the main guide member, e.g., such that the proximal end of the bridge can be inserted between the guide surfaces after performing a cut through the guide surfaces. The distal end of the bridging member can include fixation members, such as fixation pins or apertures, that allow the distal end of the bridging member to be fixated to bone. In one application, the distal end of the bridging member is fixated to a different bone than the bone the main body is fixated to such that the bridging member spans a joint. In such applications, joint spacing may be expanded or contracted by translating the shaft carried by the main body.

In addition to or in lieu of providing a bridging member, in some additional configurations, the bone cutting guide may include a secondary guide member. The secondary guide member can be positioned distally of the main guide member and may also include guide surfaces, such as opposed guide surfaces forming a channel sized and shaped to receive a cutting member. The secondary guide member may facilitate making a second bone cut distal of a location where a first bone cut is made using the main guide member.

In one example, a bone cutting guide is described that includes a support defining an inner cavity and a shaft disposed at least partially within the inner cavity, where the shaft is translatable within the inner cavity relative to the support. The bone cutting guide also includes a main guide member located on an end of the shaft, where the main guide member includes a first guide surface defining a first plane and a second guide surface defining a second plane, and where the first plane is parallel to the second plane.

In another example, a method for cutting bones is described. The method includes fixing a support to a bone and aligning a main guide member to be positioned at a location to be cut. The method further includes making a first cut at the location to be cut by inserting a cutting member through a space defined between a first guide surface of the main guide member and a second guide surface of the main guide member.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a side view of an embodiment of a bone cutting guide, with some components shown in an exploded view.

FIG. 2 is a perspective view of the bone cutting guide of FIG. 1.

FIG. 3A is a perspective view of the bone cutting guide of FIG. 1 with a bridge component attached to a main guide member.

FIGS. 3B-3D are top plan view illustrations of a bone cutting guide with different example connecting blocks.

FIG. 3E is a side plan view of a bone cutting guide and an exemplary support.

FIG. 4 is a perspective view of the bone cutting guide of FIG. 1 assembled.

FIG. 5 is another perspective view of the bone cutting guide of FIG. 4.

FIG. 6 is a further perspective view of the bone cutting guide of FIG. 4.

FIG. 7 is a perspective view of a bone cutting guide support fixed to a bone.

FIG. 8 is a top view of the bone cutting guide support fixed to the bone of FIG. 7.

FIG. 9 is a perspective view of the bone cutting guide support fixed to the bone of FIG. 7 with a location of the main guide member adjusted.

FIG. 10 is a perspective view of a bridge component attached to the main guide member of the support of FIG. 7 with a fixation structure attached to the bridge component.

FIG. 11 is a perspective view of the fixation structure pinned across a bone.

FIG. 12 is another perspective view of the pinned fixation structure of FIG. 11.

FIG. 13 is a perspective view of the assembled bone cutting guide fixed to bones.

FIG. 14 is a further perspective view of the assembled bone cutting guide of FIG. 13.

FIG. 15 shows a perspective view of the assembled bone cutting guide of FIG. 13 with a secondary guide member translated along rails of the bridge component.

FIG. 16 is an additional perspective view of the assembled bone cutting guide of FIG. 15.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and dimensions are provided for selected elements, and all other elements employ that which is known to those of ordinary skill in the field of the invention. Those skilled in the art will recognize that many of the noted examples have a variety of suitable alternatives.

Embodiments of the present invention include a bone cutting guide. In an exemplary application, the bone cutting guide can be useful during a surgical procedure, such as a bone alignment, osteotomy, fusion procedure, and/or other procedures where one or more bones are to be cut. Such a procedure can be performed, for example, on bones (e.g., adjacent bones separated by a joint or different portions of a single bone) in the foot or hand, where bones are relatively smaller compared to bones in other parts of the human anatomy. In one example, a procedure utilizing the bone cutting guide can be performed to correct an alignment between a metatarsal (e.g. a first metatarsal) and a cuneiform (e.g., a first cuneiform), such as a bunion correction. An example of such a procedure is a lapidus procedure. In another example, the procedure can be performed by modifying an alignment of a metatarsal (e.g. a first metatarsal). An example of such a procedure is a basilar metatarsal osteotomy procedure.

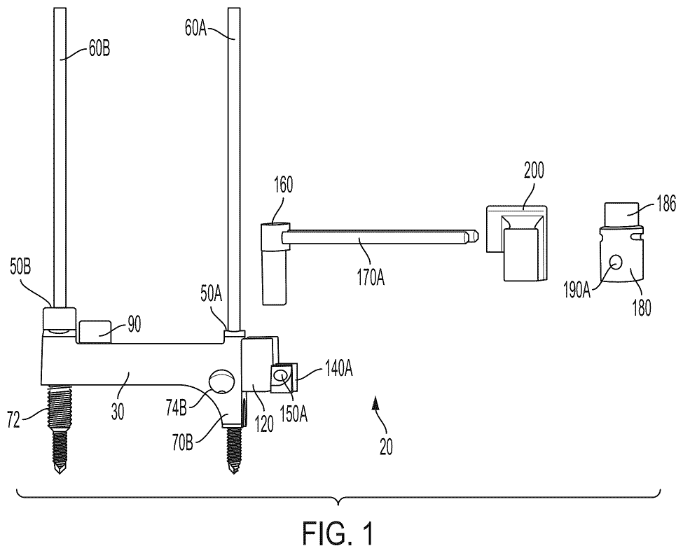

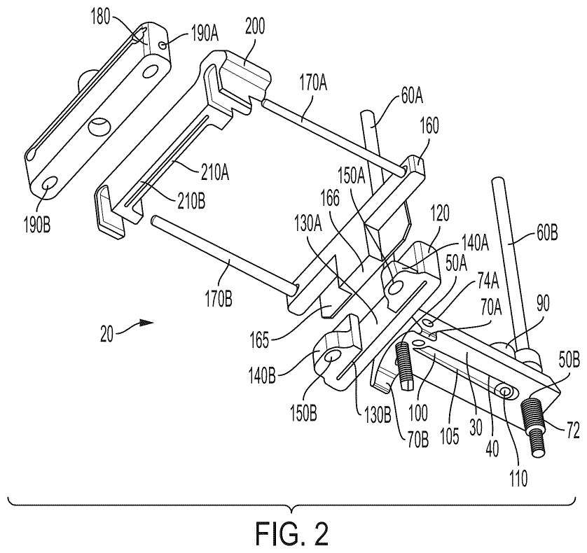

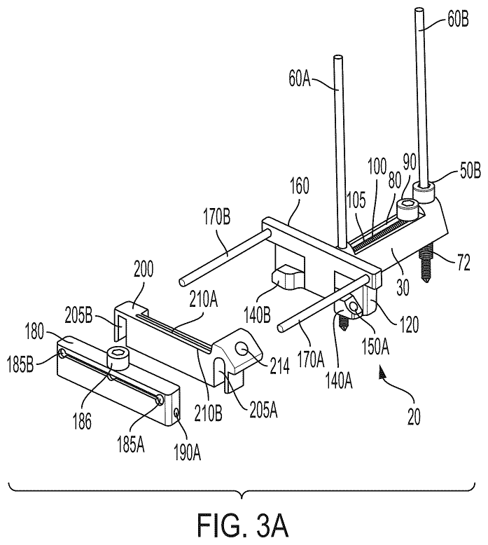

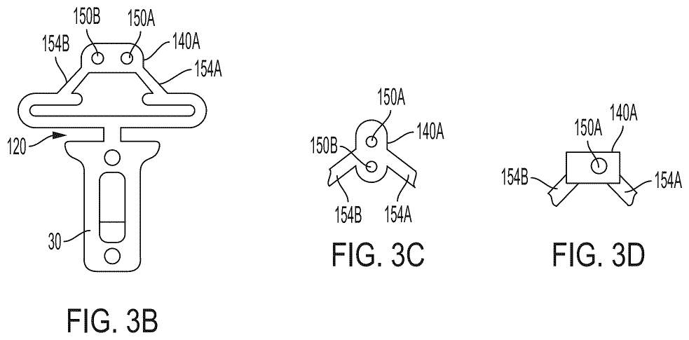

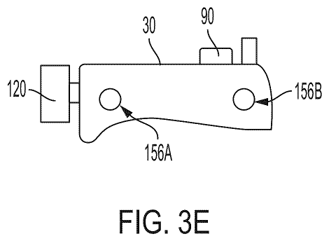

FIGS. 1, 2, and 3A show an embodiment of a bone cutting guide 20 with some components of the bone cutting guide 20 shown in an exploded view. FIG. 1 is a side view of the bone cutting guide 20, while FIGS. 2 and 3A are perspective views of the bone cutting guide 20. The bone cutting guide 20 can include a support 30 which defines an inner cavity 40 (FIG. 2). In one embodiment, the support 30 can include a first fixation aperture 50A and a second fixation aperture 50B, each of which can extend through the support 30 and receive fixation pins 60A and 60B, respectively, such that the fixation pins 60A and 60B extend through the support 30 via the fixation apertures 50A and 50B. In the embodiment shown, the fixation pins 60A and 60B have a threaded first end adapted to threadingly engage with a bone, and allow the support 30 to be translated along a longitudinal axis of both pins 60A and 60B. In the illustrated embodiments, the fixation apertures 50A and 50B are located on opposite longitudinal ends of the support 30, but in other embodiments the fixation apertures 50A and 50B can be located at various positions on the support 30.

The support 30 can further include one or more extensions 70A and/or 70B protruding generally radially out from the support 30, which may define a concave surface configured to receive a generally cylindrical bone portion. In the embodiment shown, fixation aperture 50B is provided with an extension member 72 which can be threadingly coupled to the support 30. Such an extension member 72 can be adjusted relative to the support 30 to allow the support to become parallel with a longitudinal axis of a bone, if desired. In such embodiments, the support 30 can rest on a bone via the extensions 70 A/B and extension member 72 in a position generally parallel to the bone. Fixation pin 60B may be received within an internal aperture of the extension member 72. As shown, apertures 74A and B, such as tapered apertures, may be provided proximal to extensions 70 A and B. Such apertures may extend through the support at a skewed angle relative to the longitudinal axis of the support, and may be used to engage a clamping instrument or receive fixation pins.

The support 30 can also include a slot 80 formed on at least a portion of a surface of the support 30. As illustrated in the embodiment of the cutting guide 20 shown in FIG. 3A, the slot 80 can extend in a surface of the support 30 between fixation apertures 50A and 50B. A securing component 90 can be configured to translate along the slot 80 relative to the support 30. For example, the securing component 90 can have a first end with a diameter greater than a diameter of a second opposite end, such that the first end of the securing component 90 is supported by the slot 80 (e.g., the first end has a diameter greater than a radial width of the slot 80) while the second end of the securing component 90 is positioned within the slot 80 (e.g., the second end has a diameter less than a radial width of the slot 80).

The inner cavity 40 of the support 30 can have a shaft 100 positioned at least partially within the inner cavity 40. The shaft 100 can be configured to translate within the inner cavity 40 relative to the support 30, such that an end of the shaft 100 can be made to project out from the inner cavity 40. The shaft 100 may define a slot 105 which may be aligned with the slot 80 defined by the support 30. This slot 105 may receive the pin 60A to reduce interference when the shaft 100 translates. Furthermore, the shaft 100 can include a securing aperture 110 which can be configured to receive at least a portion of the securing component 90. In one embodiment, both the second end of the securing component 90, within the slot 80, and the securing aperture 110 can be threaded to allow the securing component 90 to mate with the securing aperture 110. Such a configuration can allow the shaft 100 to be fixed, such as by compressing a surface of the support 30 that defines the slot 80, and thus prevented from translating within the inner cavity 40, relative to the support 30. In another embodiment, the securing component 90 can be threadingly engaged with the support 30 to act against the shaft 100 to prevent the shaft 100 from traveling with the cavity 40 when desired.

On an end of the shaft 100, a main guide member 120 can be disposed. In some embodiments the main guide member 120 can be integral with the shaft 100, or in other embodiments the main guide member 120 and the shaft 100 can be separate components coupled together. The main guide member 120 can have a first guide surface 130A and a second guide surface 130B, and in some embodiments the main guide member 120 can include blocks 140A and/or 140B. The first and second guide surfaces 130A and 130B can be adjacent surfaces facing one another with a space defined between the first and second guide surfaces 130A and 130B. For example, the first guide surface 130A can be a surface of the main guide member 120 immediately opposite a surface of the main guide member 120 that interfaces with the shaft 100, and the second guide surface 130B can be a surface of the main guide member 120 immediately opposite a surface of the main guide member 120 that includes blocks 140A and 140B.

In the illustrated embodiment, the second guide surface 130B contains a gap, such that the second guide surface 130B is not a single, continuous surface. In other embodiments, the second guide surface 130B can be a single, continuous surface lacking any such gap. The first guide surface 130A defines a first plane, while the second guide surface 130B defines a second plane. As shown, the first guide surface 130A and the second guide surface 130B can be configured such that the first plane is parallel to the second plane, with the space between. In further embodiments (not illustrated), the guide surfaces 130A and 130B can be configured such that the first and/or second planes are skewed.

As previously noted, a surface of the main guide member 120 can include one or more blocks 140A and 140B, either integral with the main guide member 120 or as separate components attached to the main guide member 120. As shown, the blocks 140A and 140B can be on a surface on a side of the main guide member 120 furthest from the interface with the shaft 100. In other applications, the blocks 140A and 140B can be located at various other positions on the main guide member 120. The blocks 140A and 140B can include fixation apertures 150A and 150B respectively. The fixation apertures 150A and 150B extend through the blocks 140A and 140B and provide a location for configuring additional fixation pins to, for example, position a bone or bones.

As shown in FIGS. 3B-3D, the main guide member 120 and at least one block 140A can assume other configurations. In FIG. 3B, the block 140A includes fixation apertures 150A and B and is spaced from the guide surfaces a distance via connecting flanges 154A and 154B. In the embodiment of FIG. 3B, the fixation apertures 150A and B are positioned in a line substantially parallel to the guide surfaces. In FIG. 3C, the orientation of the fixation apertures 150A and B is substantially perpendicular to the guide surfaces. In FIG. 3D, only one fixation aperture 150A is provided.

Another embodiment of a support 30 is depicted in FIG. 3E. In FIG. 3E, the support 30 has at least one (e.g., two) fixation aperture 156A and 156B formed in its side to receive fixation pins. Such apertures can also be included on the opposite side of the support (not shown). In some embodiments, the fixation apertures 156A and 156B can be positioned in a line substantially parallel with a longitudinal axis of the support, and can extend in a direction substantially perpendicular to the longitudinal axis of the support. In certain embodiments, the apertures extend at an angle, such as about 20 degrees, from vertical. In such embodiments, the support 30 can be placed on a dorsal surface and after a first cut or cuts, can be rotated about a pin extending though one of the fixation apertures 156A and 156B to rotate the support relative to the bone and first cut or cuts. The support can then be further pinned to the bone and an additional cut or cuts can be made at a desired angle relative to the first cut or cuts.

In addition to the support 30, the bone cutting guide 20 can include a bridge component 160. As shown in FIG. 3A, the bridge component 160 can attach to the main guide member 120.

In particular, in some applications of the bone cutting guide 20, the bridge component 160 can have a geometry that allows the bridge component 160 to attach to the main guide member 120 between the first and second guide surfaces 130A and 130B through an interference fit. Optionally, a locking mechanism can be provided to lock the bridge component to the main guide member, such as a locking tab, screw, pin, cam, etc. For example, the bridge component 160 may have a planar member 165 (shown in FIG. 2) that is received within the gap between the surfaces 130A and 130B and an extending block 166 (shown in FIG. 2) adapted to extend into the surface gap of 130B. In other applications, the bridge component 160 can be coupled to the main guide member 120 by any attachment mechanism, such as screws or clamps. The bridge component 160 can include rails 170A and 170B, each extending out from the bridge component 160 in a same general direction. In other embodiments, the rails 170A and 170B can extend out from the bridge component 160 at different angles.

The bone cutting guide 20 can also include in some embodiments a fixating structure 180. The fixating structure 180 can be supported on the rails 170A and 170B. For example, the fixating structure 180 can include apertures 185A and 185B to receive the rails 170A and 170B, respectively. The fixating structure 180 can be secured to the rails 170A and 170B, such that the fixating structure 180 is obstructed from translating along the rails 170A and 170B, by turning or otherwise actuating an actuator 186 of the fixating structure 180, which moves a lock (not shown) to act against the rails. Furthermore, the fixating structure 180 can also include one or more fixation apertures 190A and/or 190B. Fixation apertures 190A and 190B extend through fixating structure 180 and can be located on opposite ends of the fixating structure 180, at a skewed angle, and serve to receive fixation pins or other means for stabilizing the bone cutting guide 20 across a targeted anatomy and/or positioning a bone or bones.

Additionally, the bone cutting guide 20 can have a secondary guide member 200. The secondary guide member 200 can be supported on the rails 170A and 170B. For example, the secondary guide member 200 may include slots 205A and 205B to receive the rails 170A and 170B such that the secondary guide member 200 is supported thereon. The secondary guide member 200 can also have a third guide surface 210A and a fourth guide surface 210B. The third and fourth guide surfaces 210A and 210B can be adjacent surfaces facing one another with a space defined between the third and fourth guide surfaces 210A and 210B. In the illustrated embodiments, third and fourth guide surfaces 210A and 210B are single, continuous surfaces that do not include a gap, but in other embodiments third and/or fourth guide surfaces 210A and 210B can include a gap. The third guide surface 210A defines a third plane, while the fourth guide surface 210B defines a fourth plane. As shown, the third guide surface 210A and fourth guide surface 210B can be configured such that the third plane is parallel to the fourth plane, with the space between. In further embodiments (not illustrated), the guide surfaces 210A and 210B can be configured such that the third and/or fourth planes are skewed. Further, the third and/or fourth guide surfaces may be parallel to or skewed with respect to the first and/or second guide surfaces, such that the cutting guide can be adapted to make parallel cuts or angular cuts or cut shapes (e.g. a chevron shape). In some embodiments, the secondary guide member 200 can be locked to the rails 170A and/or 170B with a locking screw, cam, pin, etc. In the embodiment shown in FIG. 3A, an aperture 214 is provided to receive a locking mechanism and/or an accessory, such as a handle.

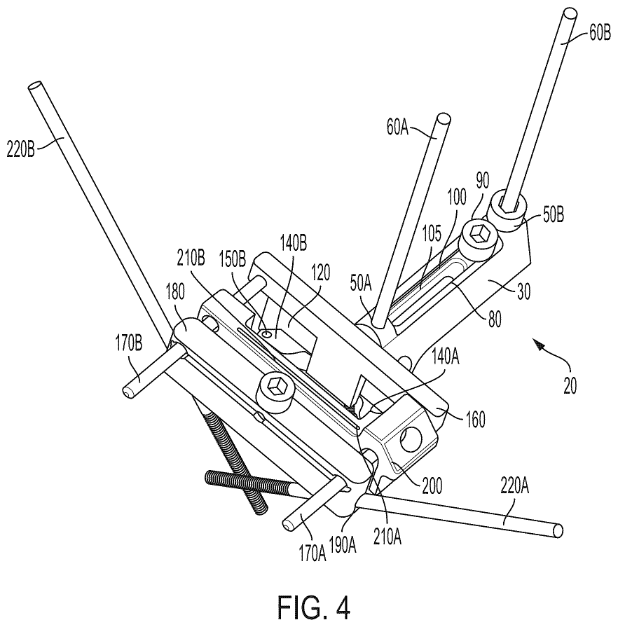

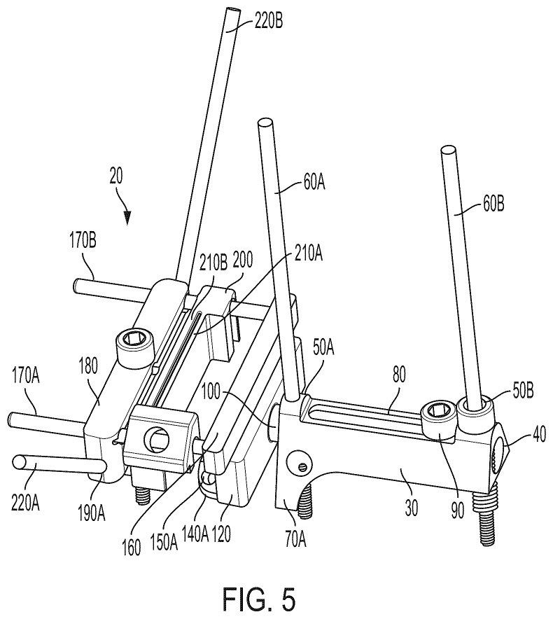

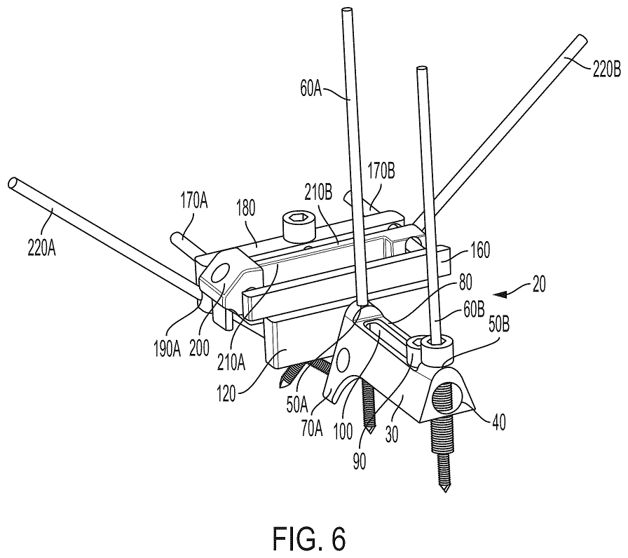

FIGS. 4-6 illustrate perspective views of the embodiment of the bone cutting guide 20, described with respect to FIGS. 1-3, as assembled. In the embodiment illustrated in FIGS. 4-6, the bridge component 160 is attached to the main guide member 120 and both the fixating structure 180 and secondary guide member 200 are supported along the rails 170A and 170B of the bridge component. In one application, the secondary guide member 200 can be supported on the rails 170A and 170B at a location along the rails 170A and 170B between the fixating structure 180 and the main guide member 120. Additionally shown in FIGS. 4-6 are fixation pins 220A and 220B received within fixation apertures 190A and 190B such that the fixation pins 220A and 220B extend through the fixating structure 180. In some applications of the bone cutting guide 20, it may be desirable to provide the fixation pins 220A and 220B at an angle other than 90 degrees relative to a top surface of the fixating structure 180 by configuring the fixation apertures 190A and 190B to extend through the fixating structure 180 at a skewed angle to guide the fixating pins 220A and 220B. Fixation pins 220A and 220B can be used, for example, for stabilizing the bone cutting guide 20 across a targeted anatomy and/or positioning a bone or bones.

Embodiments of the bone cutting guide 20 can be useful in operation for temporarily positioning a bone or bones and guiding a cutting of a bone or bones at a targeted anatomy. Bone cutting can be useful, for instance, to facilitate contact between leading edges of adjacent bones, separated by a joint, or different portions of a single bone, separated by a fracture, such as in a bone alignment and/or fusion procedure. As such, embodiments of the present invention include methods for temporarily fixing an orientation of a bone or bones, such as during a surgical procedure, and guiding cutting at desired bone locations. In the embodiments described, cuts are made to bone with respect to the cutting guide, and the bones can be positioned for an additional surgical step, such as bone plating, after the cuts have been made.

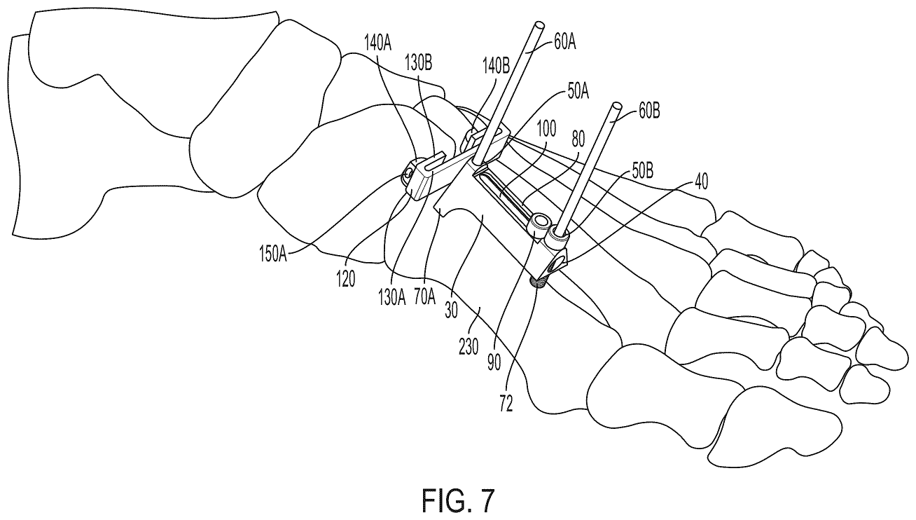

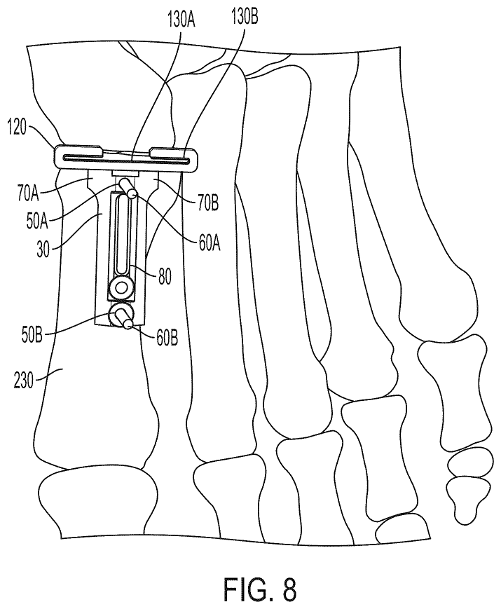

FIGS. 7-16 illustrate steps of an embodiment of a method for temporarily positioning and cutting a bone or bones using the bone cutting guide 20. Specifically, FIGS. 7 and 8 show a perspective and top view, respectively, of the support 30 fixed to a bone 230 (e.g. a first metatarsal). The support 30 is placed on the bone 230. For embodiments of the bone cutting guide 20 that include the extensions 70A and 70B, the extensions 70A and 70B can be used to at least partially straddle the bone 230 and consequently provide both greater stability to the support 30 on the bone 230 and anatomical alignment of the support 30 on a longitudinal axis of the bone 230 (e.g., the slot 80 is generally parallel to the longitudinal axis of the bone 230). Extension member 72 can be adjusted to a desired distance from support 30. Further, in some embodiments it can be desirable to align and fix the support 30 along the longitudinal axis of the bone 230 using the fixation pins 60A and 60B. The pin 60A can be inserted through the fixation aperture 50A such that an end of the pin 60A protrudes out from the fixation aperture 50A adjacent the bone 230. The pin 60A can then be fixed to the bone 230. Similarly, the pin 60B can be inserted through fixation aperture 50B and fixed on an end to the bone 230. In this manner, the support 30 can be fixed in place to and aligned along the longitudinal axis of the bone 230.

In addition to fixing the support 30 to the bone 230, the main guide member 120 can be aligned such that the main guide member 120 is positioned at a location where a bone (e.g., the bone 230) is to be cut. In one embodiment, the main guide member 120 can be positioned at the location where a bone is to be cut by appropriately positioning and fixing the support 30, e.g., such that the support 30 is fixed to the bone 230 at a location along bone 230 that results in the main guide member 120 being positioned at the location where a bone is to be cut. In some embodiments, a joint alignment blade (not shown) is inserted though the main guide member and into a joint space to help align the main guide member in a desired position. Further, in certain embodiments, a provisional fixation pin (not shown) can be inserted through a bone of interest and into an adjacent bone (e.g., though a first metatarsal and into a second metatarsal) to provide additional stability during the procedure.

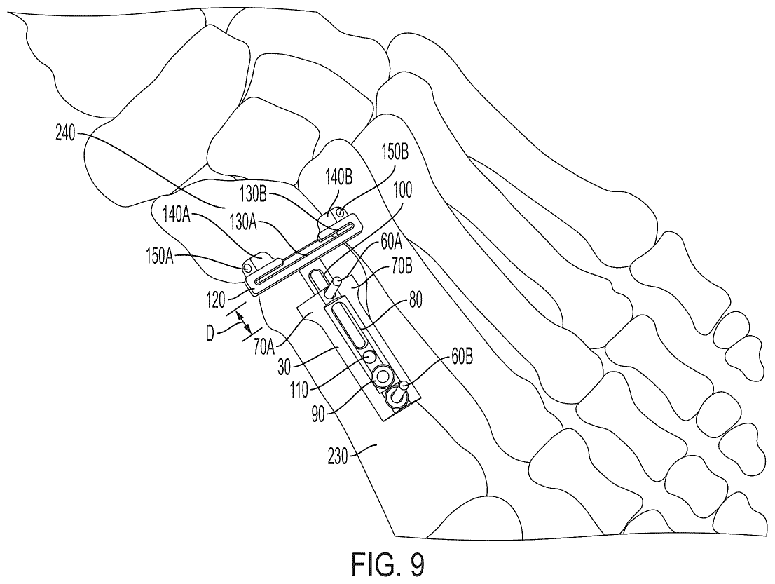

In some applications, a location of the main guide member 120 relative to the longitudinal axis of the bone 230 can be adjusted without necessitating movement of the support 30. To accomplish this, the shaft 100 at least partially within the inner cavity 40 can be translated relative to the support 30 as shown in the perspective view of FIG. 9. As shown, the main guide member 120 has been translated along the longitudinal axis of the bone 230 a distance D as a result of the shaft 100 being moved the same distance D. Once the main guide member 120 is positioned at the location to be cut, the securing component 90 can be translated along the slot 80 such that the securing component 90 is aligned with securing aperture 110. The securing component 90 can then be fixed within the securing aperture 110 such that the shaft 100 is fixed relative to the support 30.

After the main guide member 120 has been positioned at the location to be cut, a cutting member (e.g. a saw blade) can be inserted through the space defined between the first guide surface 130A and the second guide surface 130B to cut, for example, the bone 230. The guide surfaces 130A and 130B can serve to direct the cutting member to the location of the bone 230 to be cut, which in many applications can be a precise location. The break or window defined in the second guide surface 130B can assist in visualizing the portion of the bone 230 being cut.

In some embodiments, the main guide member 120 can be used to make additional cuts. In such embodiments, the securing component 90 can be loosened and the shaft 100 can be translated within the cavity to a desired position. The securing component 90 can be then be fixed within the securing aperture so the shaft is again fixed relative to the support 30. In some embodiments, fixation pins may be inserted through fixation aperture 150A and/or 150B and into the bone 230 to further stabilize the main guide member. After the main guide member 120 has been repositioned at the location to be cut, a cutting member (e.g. a saw blade) can be inserted through the space defined between the first guide surface 130A and the second guide surface 130B to cut, for example, the bone 240. The guide surfaces 130A and 130B can serve to direct the cutting member to the location of the bone 240 to be cut.

In some applications, it may be desirable to provide additional, temporary fixation of the bone 230 to allow for more accurate cutting. As best seen again in FIG. 9, blocks 140A and 140B can provide a means for additionally positioning the bone 230. Fixation pins can be inserted through the fixation aperture 150A and/or 150B and into the bone 230 to temporarily position the bone 230 and/or adjacent bone 240 for cutting. In other applications, blocks 140A and 140B may not be necessary.

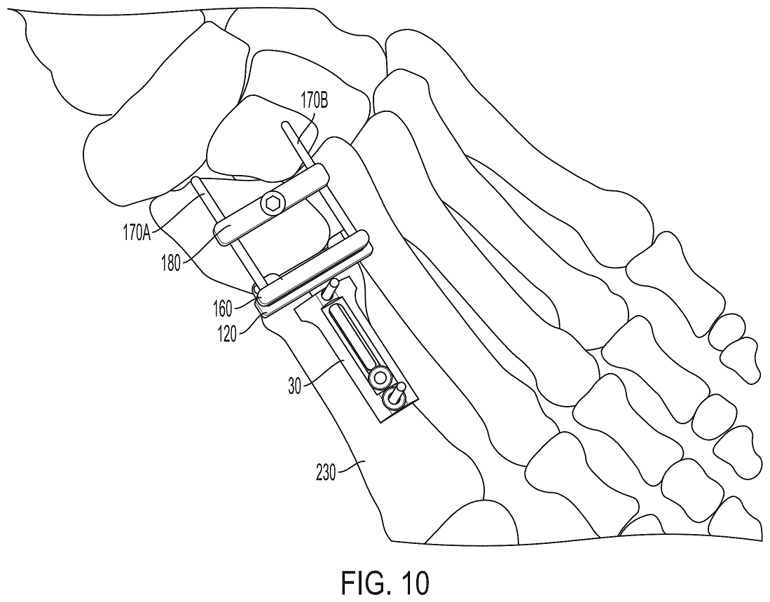

As shown in the perspective view of FIG. 10, once the bone 230 has been cut the bridge component 160 can optionally be attached to the main guide member 120. In one embodiment, the bridge component 160 can have a geometry that allows the bridge component 160 to attach to the main guide member 120 between the first and second guide surfaces 130A and 130B through an interference fit, while in other embodiments the bridge component 160 can attach to the main guide member 120 by other attachment means. The rails 170A and 170B of the bridge component 160 can be arranged such that the rails 170A and 170B extend out from the bridge component 160 on a side of the bridge component 160 opposite the support 30. The rails 170A and 170B can serve to support additional components of the bone cutting guide 20.

One such component of the bone cutting guide 20 that can be supported on the rails 170A and 170B is the fixating structure 180. FIG. 10 shows the fixating structure 180 attached to the rails 170A and 170B. In one embodiment, the fixating structure 180 can have holes or slots for receiving the rails 170A and 170B such that the fixating structure 180 can translate along the rails 170A and 170B to a desired position. The fixating structure 180, for example, can also be secured to the rails 170A and 170B in a manner that prevents translation of the fixating structure 180 when desired by actuating the actuator 186.

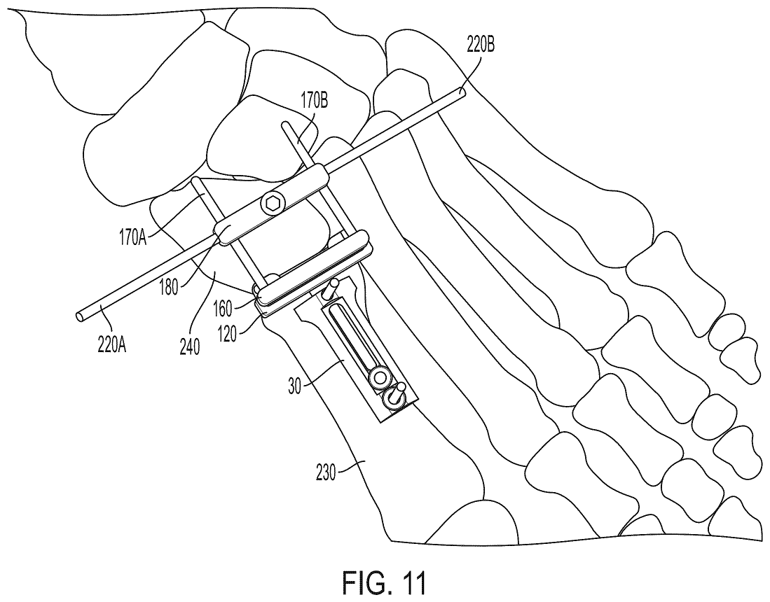

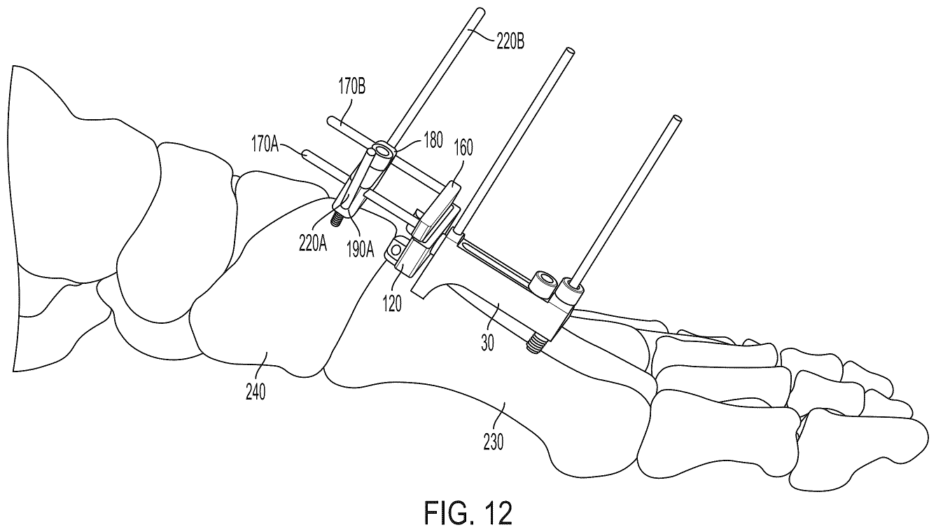

FIGS. 11 and 12 illustrate perspective views of the fixating structure 180 with the fixation pins 220A and 220B received through the fixation apertures 190A and 190B (190 B is shown in, e.g., FIG. 2). Fixation apertures 190A and 190B can be on opposite ends of the fixating structure 180 as shown. Fixation pins 220A and 220B can be fixed to a bone 240 (e.g. a first cuneiform as illustrated) to provide stability for the bone cutting guide 20 and/or to position the bone 240. After the pins 220A and 220B are set, the fixating structure 180 can be translated with respect to the rails 170A and 170B and the support 30 to a desired position to compress or expand the space between the bones 230 and 240 as needed. The position of the bones can be locked by securing the fixating structure 180 against the rails 170A and 170B. In other embodiments, such compression or expansion can be achieved by moving the shaft 100 relative to the support 30 and reengaging the securing component 90 at the new desired position.

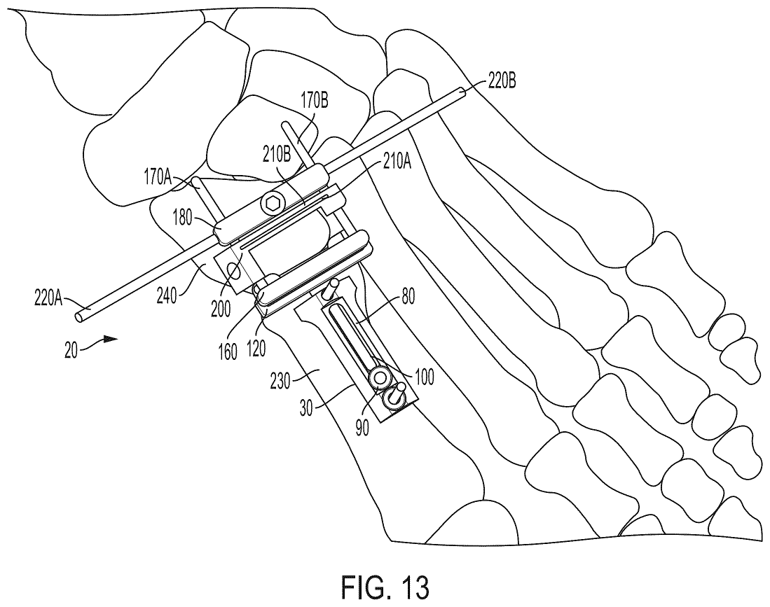

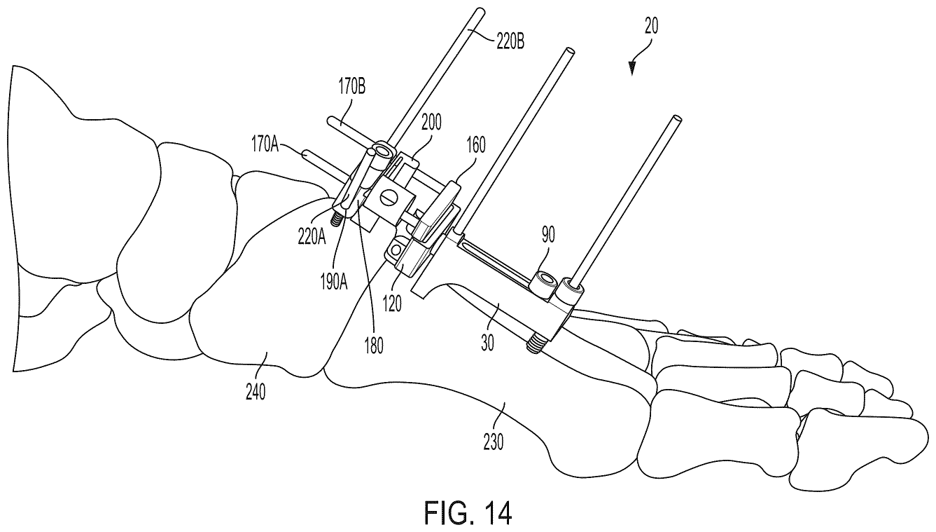

FIGS. 13 and 14 show perspective views of the bone cutting guide 20 assembled to include the secondary guide member 200. The secondary guide member 200 can be supported on the rails 170A and 170B. In one embodiment, the slots 205A and 205B of the secondary guide member 200 can receive the rails 170A and 170B such that the secondary guide member 200 can translate along the rails 170A and 170B to a desired position. As illustrated, the secondary guide member 200 can be located along the rails 170A and 170B between the fixating structure 180 and the bridge component 160.

The secondary guide member 200 can be positioned at a location where a second bone cut is to be made. A cutting member (e.g. a saw blade) can be inserted through the space defined between the third and fourth guide surfaces 210A and 210B to cut, for example, the bone 240. The guide surfaces 210A and 210B can serve to direct the cutting member to the location of the bone 240 to be cut, which in many applications can be a precise location. As illustrated, the cut made using the secondary guide member 200 (e.g. to bone 240) will be a cut that is generally parallel to the cut made using the main guide member 120. However, in other embodiments components of the bone cutting guide 20 (e.g. rails 170A and 170B) can be configured such that the cut made using the secondary guide member 200 is an angular cut (i.e. not parallel) relative to the cut made using the main guide member 120.

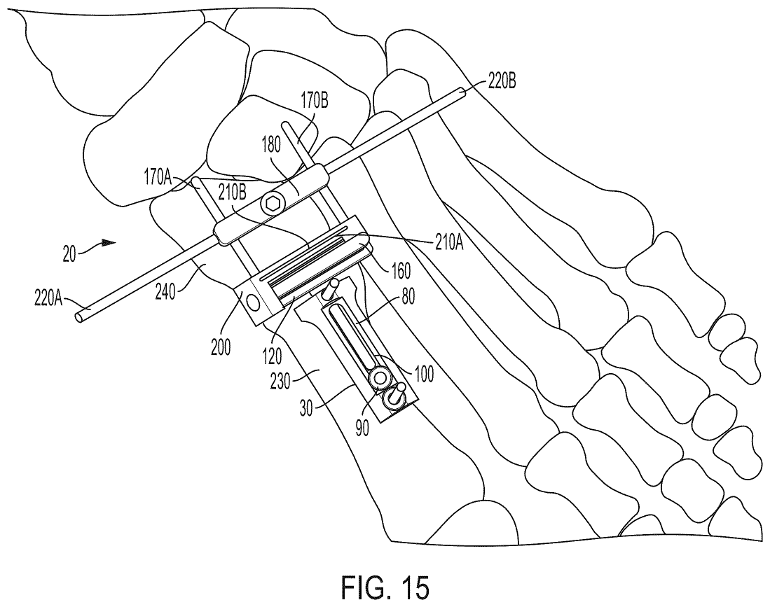

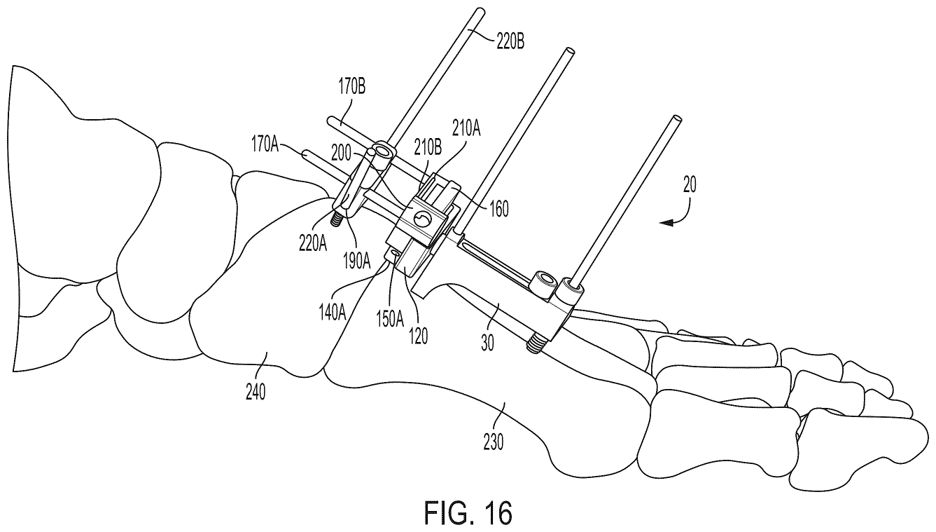

FIGS. 15 and 16 illustrate the bone cutting guide 20 as described previously, with the secondary guide member 200 translated along the rails 170A and 170B. The secondary guide member 200 can be translated along the rails 170A and 170B to precisely locate the secondary guide member 200 at the location to be cut (e.g. on bone 240). In the embodiment illustrated, the secondary guide member 200 can be shaped such that a portion of the secondary guide member 200 can overlap, or sit on top of, the bridge component 160. Such a configuration can be useful, for example, where the second cut made using the secondary guide member 200 is desired to be close to the first cut made using the main guide member 120 (e.g. portions of bones 230 and 240 interfacing at a joint).

When the bone 230 and/or bone 240 have been cut and positioned as desired, the bone cutting guide 20 can be removed. In some embodiments, the cutting guide 20 is temporarily removed from the fixation pins and cut bone is removed from the area. In certain embodiments, an autograft or other compound is delivered to the area of the bone cuts. Optionally, the guide may then be reset on the bones over the fixation pins and the shaft 100 can be translated within the cavity to adjust the relative position of the bones (e.g., to compress them together). The securing component 90 can be then be fixed within the securing aperture so the shaft is again fixed relative to the support 30. A bone plate may optionally be applied across the joint while the bones are held in the longitudinally fixed position by the cutting guide. After the plate is applied, the bone cutting guide and the fixation pins may be removed. Removing the bone cutting guide 20 can include removing all fixation pins and the support, and, in some embodiments, can include removing the bridge component, along with the fixation structure and secondary guide member 200. In certain embodiments, a second bone plate may optionally be applied across the joint. In a specific embodiment, the two bone plates are applied about 90 degrees From each other around the circumferences of the bones (e.g., at a dorsal side and a medial side).

Thus, embodiments of the invention are disclosed. Although the present invention has been described with reference to certain disclosed embodiments, the disclosed embodiments are presented for purposes of illustration, and not limitation, and other embodiments of the invention are possible. One skilled in the art will appreciate that various changes, adaptations, and modifications may be made without departing from the spirit of the invention.

1. A bunion correction method comprising:
positioning a first bone cutting guide slot over a portion of a metatarsal to be cut;
attaching the first bone cutting guide slot to the metatarsal with a first fixation pin inserted into the metatarsal and a second fixation pin inserted into the metatarsal, the first fixation pin and the second fixation pin being positioned distally of the first bone cutting guide slot;
inserting a cutting member through the first bone cutting guide slot to cut the portion of the metatarsal;
adjusting an alignment of the metatarsal relative to a cuneiform separated from the metatarsal by a joint to establish a moved position of the metatarsal;
positioning a second bone cutting guide slot over a portion of the cuneiform to be cut, the second bone cutting guide slot being attached to the metatarsal by at least the first fixation pin inserted into the metatarsal and the second fixation pin inserted into the metatarsal, the first fixation pin and the second fixation pin being positioned distally of the second bone cutting guide slot;
inserting the cutting member through the second cutting slot to cut the portion of the cuneiform; and
causing the metatarsal to fuse to the cuneiform in the moved position.

2. The method of claim 1, further comprising, after adjusting the alignment of the metatarsal relative to the cuneiform to establish the moved position of the metatarsal, lifting the first bone cutting guide slot off the first fixation pin and the second fixation pin.

3. The method of claim 1, wherein:
inserting the cutting member through the first bone cutting guide slot to cut the portion of the metatarsal comprises inserting the cutting member through the first bone cutting guide slot to cut the portion of the metatarsal without the second bone cutting guide slot being positioned over the portion of the cuneiform to be cut; and
inserting the cutting member through the second bone cutting guide slot to cut the portion of the cuneiform comprises inserting the cutting member through the second bone cutting guide slot to cut the portion of the cuneiform without the first bone cutting guide slot being positioned over the portion of the metatarsal to be cut and configured to receive the cutting instrument therethrough.

4. The method of claim 1, wherein positioning the first bone cutting guide slot over the portion of the metatarsal to be cut comprises positioning a support extending perpendicularly from the first bone cutting guide slot over a dorsal surface of the metatarsal with the support extending substantially parallel to a longitudinal axis of the metatarsal.

5. The method of claim 4, wherein attaching the bone cutting guide slot to the metatarsal with the first fixation pin inserted into the metatarsal and the second fixation pin inserted into the metatarsal comprises inserting the first fixation pin through a first fixation aperture of the support into the metatarsal and inserting the second fixation pin through a second fixation aperture of the support into the metatarsal.

6. The method of claim 1, wherein positioning the second bone cutting guide slot over the portion of the cuneiform to be cut comprises positioning a support extending perpendicularly from the second bone cutting guide slot over a dorsal surface of the metatarsal with the support extending substantially parallel to a longitudinal axis of the metatarsal.

7. The method of claim 6, wherein, when the second bone cutting guide slot is positioned over the portion of the cuneiform to be cut, the first fixation pin extends from the metatarsal through a first fixation aperture of the support and the second fixation pin extends from the metatarsal through a second fixation aperture of the support.

8. The method of claim 1, wherein attaching the first bone cutting guide slot to the metatarsal with the first fixation pin and the second fixation pin comprises:
inserting the first fixation pin extending substantially perpendicular to a dorsal surface of the metatarsal; and
inserting the second fixation pin extending substantially perpendicular to the dorsal surface of the metatarsal with the second fixation pin being substantially parallel to the first fixation pin.

9. The method of claim 1, further comprising, after positioning the second bone cutting guide slot over the portion of the cuneiform to be cut, attaching the second bone cutting guide slot to the cuneiform with a third fixation pin inserted into the cuneiform.

10. The method of claim 1, wherein the first bone cutting guide slot and the second bone cutting guide slot are configured to result in a cut end of the metatarsal being substantially parallel to a cut end of the cuneiform.

11. The method of claim 1, wherein the first bone cutting guide slot and the second bone cutting guide slot are configured to result in a cut end of the metatarsal being angled relative to a cut end of the cuneiform.

12. The method of claim 1, wherein the cutting member is a saw blade.

13. The method of claim 1, wherein inserting the cutting member through the first bone cutting guide slot and inserting the cutting member through the second bone cutting guide slot comprises inserting the cutting member through the first bone cutting guide slot to cut the portion of the metatarsal prior to inserting the cutting member through the bone cutting guide slot to cut the portion of the cuneiform.

14. The method of claim 1, wherein inserting the cutting member through the bone cutting guide slot and inserting the cutting member through the second bone cutting guide slot comprises inserting the cutting member through the first bone cutting guide slot to cut the portion of the metatarsal after inserting the cutting member through the second bone cutting guide slot to cut the portion of the cuneiform.

15. The method of claim 1, wherein:
the first bone cutting guide slot is provided between a first guide surface defining a first plane and a second guide surface defining a second plane; and
the second bone cutting guide slot is provided between a third guide surface defining a third plane and a fourth guide surface defining a fourth plane.

16. The method of claim 1, comprising:
a first guide defining the first bone cutting guide slot; and
a second guide defining the second bone cutting guide slot.

17. The method of claim 1, wherein causing the metatarsal to fuse to the cuneiform in the moved position comprises applying a bone plate across the joint.

18. The method of claim 1, further comprising compressing the metatarsal and the cuneiform together.

19. The method of claim 1, wherein the metatarsal is a first metatarsal and the cuneiform is a first cuneiform.

20. A bunion correction method comprising:
positioning a first bone cutting guide slot defined by a first guide over a portion of a metatarsal to be cut;
attaching the first guide to a dorsal surface of the metatarsal by inserting a first fixation pin through a first fixation aperture connected to the first guide and inserting a second fixation pin through a second fixation aperture connected to the first guide, wherein the first fixation aperture is offset along a length of the metatarsal from the second fixation aperture, and inserting the first fixation pin and inserting the second fixation pin comprises inserting the first fixation pin and inserting the second fixation pin extending substantially parallel to each other;
inserting a saw blade through the first bone cutting guide slot to cut the portion of the metatarsal;
adjusting an alignment of the metatarsal relative to a cuneiform separated from the metatarsal by a joint to establish a moved position of the metatarsal;
lifting the first guide off the first fixation pin and the second fixation pin;
positioning a second bone cutting guide slot defined by a second guide over a portion of the cuneiform to be cut, the second guide being attached to the metatarsal by at least the first fixation pin inserted into the metatarsal and the second fixation pin inserted into the metatarsal, and the second guide being attached to the cuneiform by at least a third fixation pin inserted into the cuneiform;
inserting the saw blade through the second cutting slot to cut the portion of the cuneiform; and
causing the metatarsal to fuse to the cuneiform in the moved position.

21. The method of claim 20, wherein:
inserting the saw blade through the first bone cutting guide slot to cut the portion of the metatarsal comprises inserting the saw blade through the first bone cutting guide slot to cut the portion of the metatarsal without the second bone cutting guide slot being positioned over the portion of the cuneiform to be cut; and
inserting the saw blade through the second bone cutting guide slot to cut the portion of the cuneiform comprises inserting the saw blade through the second bone cutting guide slot to cut the portion of the cuneiform without the first bone cutting guide slot being positioned over the portion of the metatarsal to be cut and configured to receive the saw blade therethrough.

22. The method of claim 20, wherein a support extends substantially perpendicularly from the first guide and defines the first fixation aperture and the second fixation aperture.

23. The method of claim 20, wherein inserting the first fixation pin and inserting the second fixation pin extending substantially parallel to each other comprises:
inserting the first fixation pin extending substantially perpendicular to the dorsal surface of the metatarsal; and
inserting the second fixation pin extending substantially perpendicular to the dorsal surface of the metatarsal.

24. The method of claim 20, wherein the first bone cutting guide slot and the second bone cutting guide slot are configured to result in a cut end of the metatarsal being substantially parallel to a cut end of the cuneiform.

25. The method of claim 20, wherein inserting the saw blade through the first bone cutting guide slot and inserting the saw blade through the second bone cutting guide slot comprises inserting the saw blade through the first bone cutting guide slot to cut the portion of the metatarsal prior to inserting the saw blade through the bone cutting guide slot to cut the portion of the cuneiform.

26. The method of claim 20, wherein the third fixation pin is at a skewed angle relative to the first fixation pin and the second fixation pin.

27. The method of claim 20, wherein causing the metatarsal to fuse to the cuneiform in the moved position comprises applying a bone plate across the joint.

28. The method of claim 20, further comprising compressing the metatarsal and the cuneiform together.

29. A bunion correction method comprising:
positioning a first bone cutting guide slot defined by a first guide over a portion of a first metatarsal to be cut;
attaching the first guide to a dorsal surface of the first metatarsal by inserting a first fixation pin through a first fixation aperture connected to the first guide and inserting a second fixation pin through a second fixation aperture connected to the first guide, wherein the first fixation aperture is offset along a length of the first metatarsal from the second fixation aperture, and inserting the first fixation pin and inserting the second fixation pin comprises inserting the first fixation pin and inserting the second fixation pin extending substantially parallel to each other and substantially perpendicular to the dorsal surface of the first metatarsal;
inserting a saw blade through the first bone cutting guide slot to cut the portion of the first metatarsal;
adjusting an alignment of the metatarsal relative to a first cuneiform separated from the metatarsal by a joint to establish a moved position of the first metatarsal;
lifting the first guide off the first fixation pin and the second fixation pin;
positioning a second bone cutting guide slot defined by a second guide over a portion of the first cuneiform to be cut, the second guide being attached to the first metatarsal by at least the first fixation pin and the second fixation pin, and second guide being attached to the first cuneiform by at least a third fixation pin inserted into the first cuneiform at a skewed angle relative to the first fixation pin and the second fixation pin;
inserting the saw blade through the second cutting slot to cut the portion of the first cuneiform;
compressing the first metatarsal and the first cuneiform together; and
causing the first metatarsal to fuse to the first cuneiform in the moved position.

30. The method of claim 29, wherein causing the metatarsal to fuse to the cuneiform in the moved position comprises applying a bone plate across the joint.
